(19) United States
(12) Patent Application Publication
(10) Pub. No.: US 2024/0337642 A1
(43) Pub. Date: Oct. 10, 2024

(54) SYSTEMS AND METHODS OF MEASURING MOISTURE IN SOIL USING NEUTRON SENSORS

(71) Applicant: Baker Hughes Holdings LLC, Houston, TX (US)

(72) Inventors: Ryan Conway, Brunswick, OH (US); Kevin Scott McKinny, Hudson, OH (US); Nathan D. Crowe, Steetsboro, OH (US); Matthew McPheeters, Cleveland, OH (US)

(21) Appl. No.: 18/619,970

(22) Filed: Mar. 28, 2024

Related U.S. Application Data
(60) Provisional application No. 63/457,913, filed on Apr. 7, 2023.

(51) Int. Cl. G01N 33/24; G01T 3/00; G01T 7/00

(52) U.S. Cl. CPC G01N 33/246; G01T 3/008; G01T 7/005

(57) ABSTRACT

Systems and methods for measuring soil moisture are provided. The system can include a neutron sensor configured to be provided at a sampling location and comprising one or more neutron detectors configured to detect fast neutrons and thermal neutrons at the sampling location and a computing device including at least one data processor and a memory storing instructions configured to cause the processor to perform operations including: receiving, from the neutron sensor, a first electrical signal characterizing a count of fast neutrons at the sampling location and a second electrical signal characterizing a count of thermal neutrons at the sampling location, determining a ratio of fast neutrons to thermal neutrons present at the sampling location, determining a soil moisture measurement based on the ratio and providing the soil moisture measurement.

### RELATED APPLICATION

This application claims priority under 35 U.S.C. § 119(e) to U.S. Provisional Application No. 63/457,913 filed Apr. 7, 2023, the entire contents of which are hereby expressly incorporated by reference herein.

### TECHNICAL FIELD

The subject matter described herein relates to systems and methods for measuring moisture in soil.

### BACKGROUND

Sensors, such as cosmic ray neutron sensors (CRNS) can be used to measure soil moisture. Current CRNS designs are limited in their ability to measure soil moisture over large plots of land accurately in real-time without correction or processing manipulation of the sensor data using additional inferences. As a result, soil moisture measurements may be inaccurate and can require additional time and processing to correct the inaccuracies.

### SUMMARY

In one aspect, a system for measuring moisture in soil is provided. In some aspects, the system can include a neutron sensor arranged to be provided at a sampling location and including one or more neutron detectors arranged to detect fast neutrons at the sampling location and thermal neutrons at the sampling location. The system can also include a computing device, communicatively coupled to the neutron sensor and including at least one data processor and a memory storing computer-readable instructions which, when executed by the least one data processor, cause the processor to perform operations. In some aspects, the operations performed by the processor can include receiving, from the neutron sensor, a first electrical signal characterizing a count of fast neutrons at the sampling location and a second electrical signal characterizing a count of thermal neutrons at the sampling location, determining a ratio of fast neutrons to thermal neutrons present at the sampling location, determining a soil moisture measurement based on the ratio and providing the soil moisture measurement.

In some aspects, the one or more neutron detectors can be chosen from any one of a Helium-3 detector, a Helium-4 detector, a Lithium-6 foil detector, a Lithium-glass detector, a Boron-10 detector and a Boron-trifluoride detector.

In some aspects, the one or more neutron detectors further include one or more first neutron detectors arranged to detect fast neutrons and one or more second neutron detectors arranged to detect thermal neutrons. In some aspects, the one or more first neutron detectors and the one or more second neutron detectors can be Helium-3 detectors. In some aspects, the one or more first neutron detectors can be wrapped or coated in a moderator material and shielding material and the one or more second neutron detectors can be wrapped or coated in at least the moderator material.

In some aspects, the one or more first neutron detectors can be arranged at a first location within the sampling location and the one or more second neutron detectors can include a plurality of second neutron detectors arranged at a plurality of second locations within the sampling location. In this case, the operations executed by the processor can further include receiving, from the one or more first neutron detectors, the first electrical signal, receiving, from the plurality of second neutron detectors, a plurality of second electrical signals characterizing a plurality of counts of thermal neutrons at the plurality of second locations within the sampling location, determining a plurality of ratios of fast neutrons to thermal neutrons present at the plurality of second location within the sampling location, determining one or more soil moisture measurements for the sampling location based on the plurality of ratios and providing the one or more soil moisture measurements for the sampling location.

In some aspects, the system can further include at least one voltage source coupled to the neutron sensor at a first end and arranged to provide a predetermined voltage to the neutron sensor; and at least one ground coupled to the neutron sensor and arranged to discharge electrical buildup in the one or more neutron detectors to a ground source.

In some aspects, the system can further include at least one amplifier, communicatively coupled to the neutron sensor and arranged to amplify the first electrical signal and the second electrical signal and transmit the first electrical signal and the second electrical signal to the computing device.

In some aspects, the operations executed by the at least one processor can further include receiving, from the memory, one or more calibration constants arranged to calibrate the count of fast neutrons and the count of thermal neutrons based on a plurality of environmental conditions present at the sampling location and determining, the soil moisture measurement based on the ratio and the one or more calibration constants. In some aspects, the plurality of environmental conditions include one or more of an atmospheric humidity, an atmospheric pressure, a measure of biomass, an amount of precipitation, an elevation, a latitude, a topography classification, a soil bulk density and a measure of lattice water.

In some aspects, the system can further include one or more wireless communication transceivers communicatively coupled to one or more neutron sensors and arranged to transmit the first electrical signal and the second electrical signal to the computing device wirelessly.

In some aspects, the computing device can be a mobile computing device chosen from any one of a smart-phone, a tablet, and a hand-held computing device.

In another aspect, a method for measuring moisture in soil is provided. In some aspects, the method can include providing a neutron sensor at a sampling location, the neutron sensor including one or more neutron detectors arranged to detect fast neutrons at the sampling location and thermal neutrons at the sampling location, receiving, by at least one data processor of a computing device, from the neutron sensor, a first electrical signal characterizing a count of fast neutrons at the sampling location and a second electrical signal characterizing a count of thermal neutrons at the sampling location, determining, by the at least one data processor, a ratio of fast neutrons to thermal neutrons present at the sampling location, determining, by the at least one data processor, a soil moisture measurement based on the ratio and providing the soil moisture measurement to a user interface display of the computing device.

In some aspects, the one or more neutron detectors can be chosen from any one of a Helium-3 detector, a Helium-4 detector, a Lithium-6 foil detector, a Lithium-glass detector, a Boron-10 detector and a Boron-trifluoride detector. In some aspects, the one or more neutron detectors can include one or more first neutrons detector arranged to detect fast neutrons and one or more second neutron detectors arranged to detect thermal neutrons.

In some aspects, the one or more first neutron detectors can be arranged at a first location within the sampling location and the one or more second neutron detectors can include a plurality of second neutron detectors arranged at a plurality of second locations within the sampling location. In this case, the method can further include receiving, by the at least one data processor, from the one or more first neutron detectors, the first electrical signal, receiving, by the at least one data processor, from the plurality of second neutron detectors, a plurality of second electrical signals characterizing a plurality of counts of thermal neutrons at the plurality of second locations within the sampling location, determining, by the at least one data processor, a plurality of ratios of fast neutrons to thermal neutrons based on the first electrical signal and the plurality of second electrical signals, determining one or more soil moisture measurements for the sampling location based on the plurality of ratios and providing the one or more soil moisture measurements for the sampling location to the user interface display.

In some aspects, the method can further include receiving, by the at least one data processor, from a memory of the computing device, one or more calibration constants arranged to calibrate the count of fast neutrons and the count of thermal neutrons based on a plurality of environmental conditions present at the sampling location and determining, by the at least one data processor, the soil moisture measurement based on the ratio and the one or more calibration constants. In some aspects, the plurality of environmental conditions include one or more of an atmospheric humidity, an atmospheric pressure, a measure of biomass, an amount of precipitation, an elevation, a latitude, a topography classification, a soil bulk density and a measure of lattice water.

In some aspects, the method can further include transmitting the first electrical signal and the second electrical signal from the neutron sensor to the computing device wirelessly via a wireless communication transceiver communicatively coupled to the neutron sensor.

In some aspects, the method can further include providing a predetermined voltage to the neutron sensor via a voltage source coupled to the neutron sensor; discharging electrical buildup in the one or more neutron detectors to a ground source via at least one ground coupled to the neutron sensor and transmitting, via at least one amplifier communicatively coupled to the neutron sensor, the first electrical signal and the second electrical signal to the computing device.

### DESCRIPTION OF DRAWINGS

These and other features will be more readily understood from the following detailed description taken in conjunction with the accompanying drawings, in which:

FIG. 1 is a diagram illustrating a system for measuring moisture in soil according to the subject matter provided herein;

FIG. 2 is a diagram illustrating another system for measuring moisture in soil according to the subject matter provided herein; and

FIG. 3 is a flow chart illustrating an exemplary method for measuring soil moisture using the systems described herein.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Traditionally, neutron sensors can be used to measure thermal neutrons at a sampling location. Thermal neutrons are generated when fast neutrons (high-kinetic energy neutrons typically produced during cosmic radiation and nuclear fission when cosmic rays interact with the Earth's atmosphere) undergo collisions with atoms or nuclei in a medium (e.g., soil), causing them to lose kinetic energy through elastic scattering interactions. Accordingly, the kinetic energy of thermal neutrons is comparable to the thermal energy of the surrounding atoms or molecules within the medium (on the order of millielectronvolts (mEV) to a few kiloelectronvolts (keV), depending on the temperature of the surrounding medium). When a thermal neutron is absorbed by a hydrogen nucleus (a proton), it produces a recoil proton and a gamma ray. The gamma ray can be detected by a scintillation detector, while the recoil proton can be detected by a gas-filled proportional counter. Accordingly, it is possible to estimate local soil moisture content by measuring the rate of gamma ray and proton production from the presence of thermal neutrons. However, simply measuring thermal neutrons within soil is not sufficient for accurately measuring soil moisture over a large plot of land, as these measurements are sensitive to the moisture content of the soil local to the moisture sensor without being able to measure changes in factors like solar radiation and how that affects the moisture content of a larger plot of soil proximal to the sensor. Traditionally, this issue is solved by correcting the local measurements with correction factors derived from sample data from databases (e.g., the International Neutron Monitor Database), where users can make inferences regarding environmental conditions at the sampling location (e.g., measures of solar activity, atmospheric pressure, water-vapor in air, humidity, etc.). However, this traditional inferential method can lead to errors in the resultant soil moisture measurements, as the actual and dynamic environmental conditions at the sampling location are overlooked.

The systems and methods described herein address the aforementioned shortcomings by providing one or more customized neutron sensors configured to measure both thermal neutron flux and fast neutron flux at a sampling location. As described above, fast neutrons are high-kinetic energy neutrons typically produced during cosmic radiation and nuclear fission when cosmic rays interact with the Earth's atmosphere. Accordingly, by measuring fast neutron flux at the sampling location, the systems and methods described herein are able to measure factors like changes in solar activity, which affect soil moisture and are overlooked by traditional methods. When fast neutrons interact with the nuclei of atoms (e.g., hydrogen) in the soil, they can produce a number of secondary neutrons, the number of which depend on the hydrogen content of the soil. By directly measuring fast and thermal neutrons, the systems and methods described herein are capable of accurately measuring soil moisture content by comparing and determining a ratio between fast neutron flux and thermal neutron flux at a sampling location without having to infer the local fast neutron flux based on sample data in databases, as described above.

Advantageously, the systems and methods described herein can generate more accurate soil moisture measurements compared to existing neutron sensor systems by measuring moisture content in soil using both fast neutrons and thermal neutrons. The systems and methods herein can enable more accurate generation of soil moisture measurements over a large area (e.g., on the order of 300 meters around the sensing areas), with reduced processing time, additional data sources, and computing power compared to existing neutron sensor systems. The system herein can be deployed in remote locations with limited water resources to provided efficient and accurate soil moisture measurements for agricultural, environmental, or conservation purposes. By providing accurate soil moisture readings, the systems and methods described herein allow users (e.g., farmers) to optimize resources for farming. For example, in dry regions where drought often occurs, causing water prices to rise and making crop yield more difficult, the systems and methods described herein allow for users to accurately understand the soil moisture levels across wide ranges of their land to better inform them in making decisions around when to water crops to avoid crop loss due to both over and under watering.

Embodiments of sensor and method of operation are discussed herein in regard to use in an agricultural environment. However, embodiments of the disclosure can be employed for sensing moisture, in any application or environment without limitation.

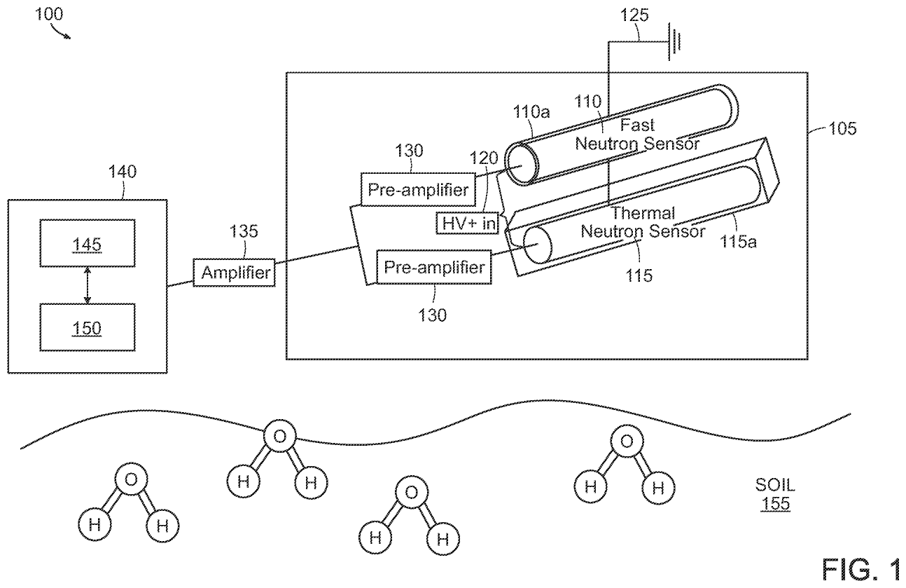

FIG. 1 is a diagram illustrating an embodiment of a system 100 including one or more neutron sensors 105 configured to determine a moisture content in soil 155 at a sampling location according to the subject matter provided herein. As shown in FIG. 1, the neutron sensor 105 can include one or more neutron detectors 110, 115 configured to detect fast neutrons and thermal neutrons in a sampling location/region. In some aspects, as shown in FIG. 1, the neutron sensor 105 can be positioned above the soil 155 at a sampling, for example, by being directly placed on the soil or being mounted to a tripod, a stake, a pole or the like that is place on or inserted into the soil. Additionally, in some aspects, the one or more neutron detectors 110, 115 of the neutron sensor 105 can be positioned at disparate locations across the sampling location/region, as discussed in greater detail below. As fast neutrons penetrate the soil 155 and undergo interactions with soil particles, water molecules, and other nuclei within the soil 155, the flux of fast neutrons detected by the one or more neutron detectors 110, 115 is influenced by the scattering and moderation processes that occur as the neutrons interact with the soil. For example, higher soil moisture content enhances the moderation and scattering of fast neutrons due to increased collisions with hydrogen atoms in soil water molecules which results in a decrease in the detected flux of fast neutrons, while a decrease in soil moisture results in an increase in the detected flux. By monitoring changes in the flux of fast neutrons over time, variations in soil moisture content can be determined. Thermal neutrons, on the other hand, are produced from the moderation of fast neutrons and reach thermal equilibrium with the surrounding environment. The flux of thermal neutrons can also be detected by the one or more neutron detectors 110, 115 and can provide additional information about soil moisture content. For example, higher soil moisture enhances the thermalization of neutrons, resulting in a decrease in the detected flux, while a decrease in soil moisture results in an increase in the detected flux.

For example, In some aspects, the neutron sensor 105 can include a single neutron detector 110 or 115 configured to measure both fast neutrons and thermal neutrons, however, in some aspects, the neutron sensor 105 can include a first neutron detector 110 configured to detect fast neutrons and a second neutron detector 115 configured to detect thermal neutrons, as described in greater detail below. In some aspects, the one or more neutron detectors 110, 115 can be chosen from any one of Helium gas filled proportional counters (3He Proportional Counters), Helium-4 detectors (4He recoil detectors), Lithium-6 Foil neutron detectors, Lithium-glass detectors, Boron-10 proportional counters and Boron-trifluoride (BF3) sensors. In some aspects, the one or more neutron detectors 110, 115 can include moderator materials to reduce energy of neutrons by scattering them and shielding materials to enhance absorption and to prevent further scattering. Accordingly, the use of moderator and shielding materials can enhance the detector's sensitivities to thermal and fast neutrons. For example, in some aspects, the first neutron detector 110 configured to detect fast neutrons can be wrapped or coated in a first material 110a or a combination of first materials including a moderator material (e.g., high-density polyethylene) and a shielding material (e.g., cadmium). In some aspects, the second neutron detector 115 configured to detect thermal neutrons can be wrapped or coated in a second material 115a or a combination of second materials. For example, the second materials 115a can include a moderator material (e.g., high-density polyethylene). It should be noted that, in some aspects, the first and/or second neutron detectors 110, 115 may be bare (not coated or wrapped in materials 110a, 110b, respectively).

As further shown in FIG. 1, in some aspects, each neutron detector can be coupled to a high voltage source 120, a ground 125, and one or more pre-amplifiers 130. Electrical signals generated by the first neutron detector 110 and the second neutron detector 115, respectively, can be provided to an amplifier 135 and to a computing device 140, through the preamplifiers 130. In some embodiments, the computing device 140 can include a one or more processors 145 and a memory 150 storing data and computer-readable instructions which, when executed by the least one data processor, cause the processor to perform operations which are described in greater detail below. The computing device 140 can be configured to determine a soil moisture measurement based on the electrical signals output by the neutron sensor 105. In some aspects, the system 100 can further include an additional data logging system (not shown) communicatively coupled to the computing device 140 and configured to store data received from the one or more neutron sensors 105.

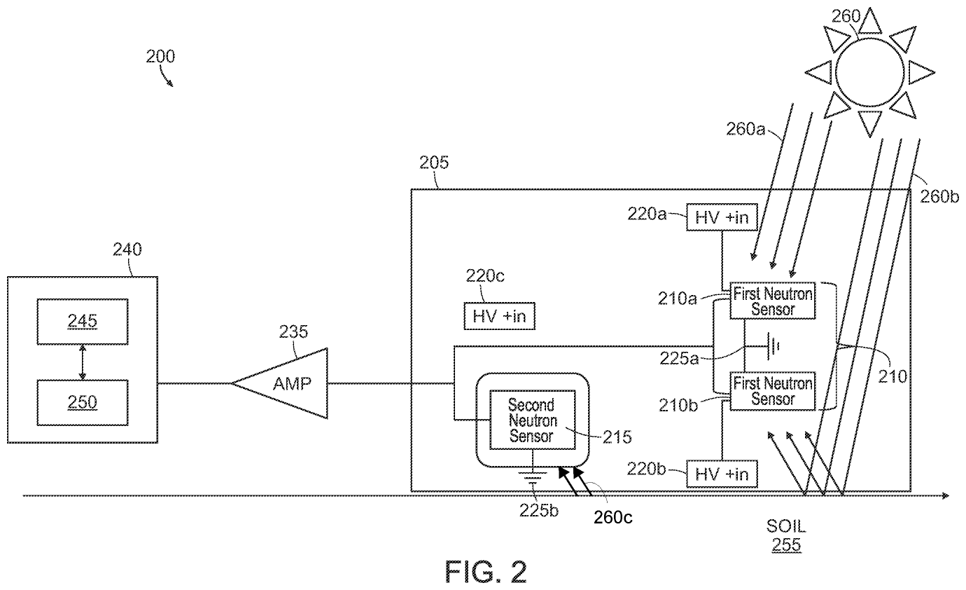

FIG. 2 is a diagram illustrating an embodiment of a system 200 including another neutron sensor 205 according to the subject matter provided herein, configured to determine a moisture content in soil 255 at a sampling location. The system 200 shown in FIG. 2 includes similar components and perform similar functions as the system 100 of FIG. 1 except where noted otherwise. As shown in FIG. 2, the neutron sensor 205 can include a first neutron detector 210 including a plurality of first neutron detectors 210a, 210b configured to detect fast neutrons 260a, 260b at the sampling location or across a larger sampling region containing the sampling location. In some aspects, by having a plurality of first neutron detectors 210a, 210b one or more of the plurality of first neutron detectors 210a, 210b can be positioned to detect fast neutrons 260a that are directly incident from the atmosphere and one or more of the plurality of first neutron detectors 210a, 210b can be positioned to detect fast neutrons 260b that are deflected off of materials (e.g., the ground) without losing a significant amount of energy. The system 200 can also include one or more second neutron detectors 215 configured to detect thermal neutrons 260c produced by higher energy neutrons (e.g., fast neutrons) colliding with atoms (e.g., hydrogen) or nuclei in the atmosphere and/or the soil, causing them to lose kinetic energy. While the system 200 of FIG. 2 only illustrates a single second neutron detector 215, it should be noted that the system 200 can include a plurality of second neutron detectors 215 configured to detect thermal neutrons 260c across the sampling location/region to improve the systems sensitivity and/or increase a range and accuracy of the resultant soil moisture measurement. For example, similarly to as described above in reference to FIG. 1, in some aspects, the sensor 205 can include a single first neutron detector 210a, 210b or a plurality of first neutron detectors 210a, 210b provided at one location within a larger sampling region and configured to detect fast neutrons 260a, 260b for the sampling region and a plurality of second neutron detectors 215 provided at in a distributed manner across a plurality of sampling locations within the larger sampling region and configured to detect thermal neutrons 260c at the plurality of sampling locations. In this case, the system 200 can use first electrical signals from the first neutron detectors 210a, 210b characterizing a count of fast neutrons 260a, 260b for the sampling region and second electrical signals from the plurality of distributed second neutron detectors 215 characterizing a count of thermal neutrons at the sampling location to determine a plurality of ratios of the fast neutron count to the plurality of thermal neutron counts and determine one or more soil moisture measurements based on the plurality of ratios.

In some aspects, the plurality of first neutron detectors 210a, 210 and the one or more second neutron detectors 215 can be chosen from any one of the detector types described above in reference to FIG. 1. In some aspects, each neutron detector 210a, 210b of the first neutron detector 210 can be coupled to a high voltage source 220a, 220b, respectively, and a ground 225a. In some embodiments, the high voltage sources 220a, 220b can be the same voltage source. In some embodiments, the second neutron detector 215 can also be coupled to a high voltage source 220c. The second neutron detector 215 can also be coupled to a ground 225b. In some embodiments, the grounds 225a and 225b can be the same ground. In some aspects, electrical signals provided by the pair 210a, 210b of first neutron detectors 210 and the second neutron detector 220 can be provided to an amplifier 235 and to a computing device 240. In some embodiments, the computing device 240 can include at least one processor 245 and a memory 250, similarly to as described above. The computing device 240 can be configured to determine a soil moisture measurement based on the electrical signals output by the neutron sensor 205, as described in greater detail below.

In some embodiments, the neutron sensor 105 or 205 can be configured in a network of sensors. In the network of sensors, a plurality of neutron sensors 105 and/or 205 can be arranged in a distributed manner across an area or sampling region and can be communicatively coupled to an amplifier and computing device. In some embodiments, the neutron sensors 105 and/or 205 can include wireless communication transceivers configured to communicate wirelessly with the computing device. In some embodiments, the system 100 and/or 200 can include a mobile computing platform. For example, the computing device can be a mobile computing device, such as a smart-phone, tablet, or hand-held computing device configured to receive the electrical signals from the neutron sensors 105 and/or 205. By providing a plurality of neutron sensors 105 and/or 205 across an area or sampling region, the system can acquire a plurality fast and thermal neutron measurements across a variety of locations. Using the plurality fast and thermal neutron measurements, the system can determine a plurality of ratios of fast neutrons to thermal neutrons present at the plurality of locations and determine a soil moisture measurements for the each of the plurality of locations as well as a soil moisture measurement for the entire sampling region based on the plurality of ratios. In some aspects, the soil moisture measurement for the entire sampling region can be determined by averaging the soil moisture measurements across each of the locations. For example, in some aspects, the soil moisture measurement for the entire sampling region can be determined by performing weighted averaging or density-based weighted averaging.

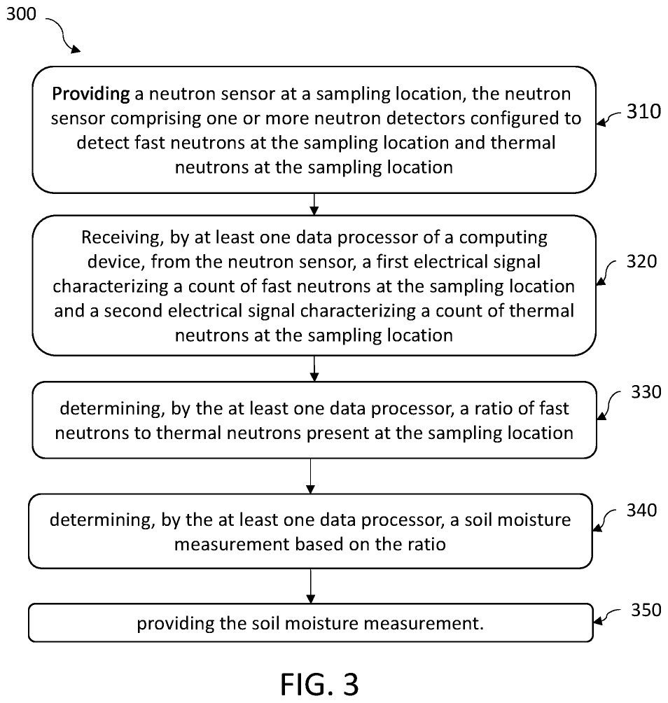

FIG. 3 is a flow chart illustrating an embodiment of a method 300 for measuring soil moisture using the systems of FIG. 1 according to the subject matter provided herein. The method 300 can include a step 310, of providing a neutron sensor at a sampling location, the neutron sensor comprising one or more neutron detectors configured to detect fast neutrons at the sampling location and thermal neutrons at the sampling location. In some aspects, the neutron sensor can be similar to neutron sensor 105 or 205 described in relation to FIGS. 1 and 2. Accordingly, in some aspects, the neutron sensor can include a single neutron detector configured to measure both fast neutrons and thermal neutrons. In some aspects, the neutron sensor can include one or more first neutron detectors configured to detect fast neutrons and one or more second neutron detectors configured to detect thermal neutrons. In some aspects, the neutron sensor can be provided above soil at the sampling location, for example, by being directly placed on the soil or being mounted to a tripod, a stake, a pole or the like, that is place on or inserted into the soil. The neutron sensor can be configured to acquire neutron flux measurements from the one or more neutron detectors and generate a first electrical signal characterizing a count of fast neutrons from an atmosphere at the sampling location and a second electrical signal characterizing a count of thermal neutrons in soil at the sampling location.

The method 300 can also include a step 320 of receiving, by at least one data processor of a computing device, from the neutron sensor, the first electrical signal characterizing the count of fast neutrons at the sampling location and the second electrical signal characterizing the count of thermal neutrons at the sampling location. In some aspects, the first electrical signal and the second electrical signal can be transmitted from the neutron sensor to the computing device wirelessly via a wireless communication transceiver communicatively coupled to the neutron sensor. In some aspects, the neutron sensor can further include at least one amplifier communicatively coupled to the neutron sensor and configured to amplify the first electrical signal and the second electrical signal as it is transmitted to the computing device.

In some aspects, step 320 can further include receiving, by the at least one processor, one or more calibration constants configured to calibrate the count of fast neutrons and the count of thermal neutrons based on a plurality of environmental conditions present at the sampling location. As described above, there is an analytical relationship between the thermal neutron flux, fast neutron flux and the amount of moisture in the soil around the sensor. In some aspects, the ratio of thermal neutrons to fast neutrons may be related moisture linearly, or through a higher order function. Accordingly, in some aspects, the method can also include an initial step of calibrating one or more parameters or aspects of the neutron sensors or the system 100. Calibrating one or more parameters or aspects of the system 100 can include collecting data from the sensors described herein (e.g., neutron sensor 105, 205) and/or via other means of data collection in a representative set of environmental conditions of the sampling location and determining relationship between the thermal neutron flux, fast neutron flux and the amount of moisture in the soil around the sensor as well as determining any constants of proportionality or higher order terms that may be required for calibration. For example, in some aspects, the initial calibration step of determining any constants of proportionality, environmental corrections, and/or higher order terms that may be required for calibration can include, but is not limited to, collecting data from other sensors regarding one or more of changes in atmospheric pressure/humidity. Additionally, the initial calibration step of determining any constants of proportionality, environmental corrections, and/or higher order terms that may be required for calibration can include, but is not limited to, collecting data regarding the presence of biomass in/above the soil, an amount of precipitation, changes in elevation/latitude/topography, changes in soil bulk density and the presence of lattice water in the soil.

Changes in atmospheric pressure affect the frequencies at which fast neutrons to collide with particles in the atmosphere which can effect the density of fast neutrons near the surface of the soil, which can impact soil moisture measurements determined using neutron sensor as described herein. Additionally, slow neutrons are measured when fast neutrons collide with atoms of low atomic weight (e.g., hydrogen). Since water contains most of the hydrogen in soil, changes in atmospheric humidity can affect soil moisture measurements determined using neutron sensor as described herein. Further, changes in solar activity can influence temperature and precipitation patterns, which affect soil moisture. Accordingly, by collecting data regarding changes in atmospheric pressure/humidity at the sampling location, the systems and methods described herein can accurately determine the analytical relationships these changes and the fast and thermal neutron flux to determine soil moisture content more accurately.

Additionally, the presence of biomass can influences soil moisture through its effects on evapotranspiration. For example, more biomass within soil can lead to increased transpiration, which draws moisture from the soil. Biomass can also act as mulch, reducing evaporation from the soil surface and preserving moisture. In some aspects, biomass in a region of soil can be determined by physically separating and quantifying different components of biomass within a soil sample, by taking soil respiration measurements, by fumigating soil samples with chloroform to lyse microbial cells and analyzing the released carbon and nitrogen, however other methods of determining biomass are also realized. Precipitation directly affects moisture, with higher precipitation levels generally leading to increased soil moisture and lower precipitation levels leading to dryer soil. Elevation, latitude, and topography affect factors such as temperature, precipitation patterns, and slope, which in turn influence soil moisture. For example, higher elevations and latitudes often experience cooler temperatures and may receive more precipitation, leading to higher soil moisture levels. Topography can affect water runoff and drainage patterns, impacting soil moisture distribution. Soil bulk density (the mass of soil per unit volume) and can impact soil moisture retention with higher soil bulk density generally reducing pore space available for water storage, leading to decreased soil moisture levels and lower bulk density allowing for greater water retention capacity. These changes in soil porosity can also affect soil moisture measurements determined using neutron sensor as described herein. Lattice water (water molecules that are bound within the crystal lattice structure of certain minerals in the soil) is another source of hydrogen within soil, which is important to consider when making soil moisture measurements using neutron sensor as described herein. Increases in lattice water content can enhance soil moisture retention, while decreases may lead to drier soil conditions. Accordingly, by collecting data regarding biomass, precipitation, elevation, latitude, topography, soil bulk density and lattice water content at the sampling location, the systems and methods described herein can accurately determine the analytical relationships these environmental factors and the fast and thermal neutron flux to determine soil moisture content more accurately. However, it should be noted the necessity for environmental corrections, or the overall magnitude of the corrections can be reduced by taking local measurements of the cosmic neutron flux within the soil as described above.

The method 300 can also include a step 330 of determining, by the at least one processor, a ratio of fast neutrons to thermal neutrons present at the sampling location based on the first electrical signal and the second electrical signal. For example, based on the first electrical signal and second electrical signal output by the first neutron detector and the second neutron detector of the neutron sensor 105, a ratio of the count of fast neutrons and the count of thermal neutrons can be determined by the computing device 140 that is indicative of a ratio of fast neutrons to thermal neutrons present at the sampling location.

The method 300 can also include a step 340 of determining, by the at least one processor, a soil moisture measurement based on the ratio of fast neutrons to thermal neutrons present at the sampling location. In some embodiments, the one or more calibration constants described above can be used to convert ratio to moisture content. Additional environmental corrections for changes in atmospheric pressure/humidity, the presence of biomass in/above the soil, an amount of precipitation, changes in elevation/latitude/topography, changes in soil bulk density and the presence of lattice water in the soil can be determined based on data collection from other sensors and/or through experimental findings or simulation and may also be applied as described above.

The method 300 can also include a step 350 of providing the soil moisture measurement. For example, in some embodiments the computing device 140, can further include a display configured to display the soil moisture measurement. Additionally, the soil moisture measurement can be provided for storage in the memory and/or the data logging system as described herein.

The improved system and methods described herein address the technical problem of accurately determining soil moisture measurements in real-time without additional post-processing or computing resources. Further, the methods described herein account for the contribution of fast neutron rays and thermal neutron rays in soil moisture determination, which is not previously known or implemented in a neutron sensor system. Additionally, the systems, sensors and methods herein can enable rapid deployment of the systems (e.g., systems 100, 200) for use in distributed monitoring applications in environments where water conservation and soil management is important, such as determination and/or monitoring of crop watering schedules.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. In some embodiments, the computer and/or microprocessor can be configured in a cloud-computing environment, a containerized computing environment, a distributed computing environment, or the like. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A system comprising:
a neutron sensor configured to be provided at a sampling location and comprising one or more neutron detectors configured to detect fast neutrons at the sampling location and thermal neutrons at the sampling location; and
a computing device, communicatively coupled to the neutron sensor and including at least one data processor and a memory storing computer-readable instructions which, when executed by the least one data processor, cause the processor to perform operations including:
receiving, from the neutron sensor, a first electrical signal characterizing a count of fast neutrons at the sampling location and a second electrical signal characterizing a count of thermal neutrons at the sampling location;
determining a ratio of fast neutrons to thermal neutrons present at the sampling location;
determining a soil moisture measurement based on the ratio; and
providing the soil moisture measurement.

2. The system of claim 1, wherein the one or more neutron detectors are chosen from any one of a Helium-3 detector, a Helium-4 detector, a Lithium-6 foil detector, a Lithium-glass detector, a Boron-10 detector and a Boron-trifluoride detector.

3. The system of claim 2, wherein the one or more neutron detectors further comprise:
one or more first neutron detectors configured to detect fast neutrons; and
one or more second neutron detectors configured to detect thermal neutrons.

4. The system of claim 3, wherein the one or more first neutron detectors and the one or more second neutron detectors are Helium-3 detectors.

5. The system of claim 3, wherein the one or more first neutron detectors are wrapped or coated in a moderator material and a shielding material and the one or more second neutron detectors are wrapped or coated in at least the moderator material.

6. The system of claim 3, wherein the one or more first neutron detectors are arranged at a first location within the sampling location and the one or more second neutron detectors comprise a plurality of second neutron detectors arranged at a plurality of second locations within the sampling location and the operations executed by the at least one processor further comprise:
receiving, from the one or more first neutron detectors, the first electrical signal;
receiving, from the plurality of second neutron detectors, a plurality of second electrical signals characterizing a plurality of counts of thermal neutrons at the plurality of second locations within the sampling location;
determining a plurality of ratios of fast neutrons to thermal neutrons present at the plurality of second location within the sampling location;
determining one or more soil moisture measurements for the sampling location based on the plurality of ratios; and
providing the one or more soil moisture measurements for the sampling location.

7. The system of claim 1, further comprising:
at least one voltage source coupled to the neutron sensor at a first end and configured to provide a predetermined voltage to the neutron sensor; and
at least one ground coupled to the neutron sensor and configured to discharge electrical buildup in the one or more neutron detectors to a ground source.

8. The system of claim 1, further comprising:
at least one amplifier, communicatively coupled to the neutron sensor and configured to amplify the first electrical signal and the second electrical signal and transmit the first electrical signal and the second electrical signal to the computing device.

9. The system of claim 1, wherein the operations executed by the at least one processor further comprises:
receiving, from the memory, one or more calibration constants configured to calibrate the count of fast neutrons and the count of thermal neutrons based on a plurality of environmental conditions present at the sampling location; and
determining the soil moisture measurement based on the ratio and the one or more calibration constants.

10. The system of claim 9, wherein the plurality of environmental conditions include one or more of an atmospheric humidity, an atmospheric pressure, a measure of biomass, an amount of precipitation, an elevation, a latitude, a topography classification, a soil bulk density and a measure of lattice water.

11. The system of claim 1, further comprising:
one or more wireless communication transceivers communicatively coupled to one or more neutron sensors and configured to transmit the first electrical signal and the second electrical signal to the computing device wirelessly.

12. The system of claim 1, wherein the computing device is a mobile computing device chosen from any one of a smart-phone, a tablet, and a hand-held computing device.

13. A method comprising:
providing a neutron sensor at a sampling location, the neutron sensor comprising one or more neutron detectors configured to detect fast neutrons at the sampling location and thermal neutrons at the sampling location;
receiving, by at least one data processor of a computing device, from the neutron sensor, a first electrical signal characterizing a count of fast neutrons at the sampling location and a second electrical signal characterizing a count of thermal neutrons at the sampling location;
determining, by the at least one data processor, a ratio of fast neutrons to thermal neutrons present at the sampling location;
determining, by the at least one data processor, a soil moisture measurement based on the ratio; and
providing the soil moisture measurement to a user interface display of the computing device.

14. The method of claim 13, wherein the one or more neutron detectors are chosen from any one of a Helium-3 detector, a Helium-4 detector, a Lithium-6 foil detector, a Lithium-glass detector, a Boron-10 detector and a Boron-trifluoride detector.

15. The method of claim 14, wherein the one or more neutron detectors includes one or more first neutrons detector configured to detect fast neutrons and one or more second neutron detectors configured to detect thermal neutrons.

16. The method of claim 15, wherein the one or more first neutron detectors are arranged at a first location within the sampling location and the one or more second neutron detectors comprise a plurality of second neutron detectors arranged at a plurality of second locations within the sampling location, the method further comprising:
receiving, by the at least one data processor, from the one or more first neutron detectors, the first electrical signal;
receiving, by the at least one data processor, from the plurality of second neutron detectors, a plurality of second electrical signals characterizing a plurality of counts of thermal neutrons at the plurality of second locations within the sampling location;
determining, by the at least one data processor, a plurality of ratios of fast neutrons to thermal neutrons based on the first electrical signal and the plurality of second electrical signals;
determining one or more soil moisture measurements for the sampling location based on the plurality of ratios; and
providing the one or more soil moisture measurements for the sampling location to the user interface display.

17. The method of claim 13, further comprising:
receiving, by the at least one data processor, from a memory of the computing device, one or more calibration constants configured to calibrate the count of fast neutrons and the count of thermal neutrons based on a plurality of environmental conditions present at the sampling location; and
determining, by the at least one data processor, the soil moisture measurement based on the ratio and the one or more calibration constants.

18. The method of claim 17, wherein the plurality of environmental conditions include one or more of an atmospheric humidity, an atmospheric pressure, a measure of biomass, an amount of precipitation, an elevation, a latitude, a topography classification, a soil bulk density and a measure of lattice water.

19. The method of claim 13, further comprising:
transmitting the first electrical signal and the second electrical signal from the neutron sensor to the computing device wirelessly via a wireless communication transceiver communicatively coupled to the neutron sensor.

20. The method of claim 13, further comprising:
providing a predetermined voltage to the neutron sensor via a voltage source coupled to the neutron sensor;
discharging electrical buildup in the one or more neutron detectors to a ground source via at least one ground coupled to the neutron sensor; and
transmitting, via at least one amplifier communicatively coupled to the neutron sensor, the first electrical signal and the second electrical signal to the computing device.
